(19) United States
(12) Patent Application Publication
(10) Pub. No.: US 2003/0211177 A1
(43) Pub. Date: Nov. 13, 2003

(54) Method and composition for treatment and prevention of alopecia

(76) Inventor: Edwin H Pineda, Portage, WI (US)

Correspondence Address: PRICE HENEVELD COOPER DEWITT & LITTON, 695 KENMOOR, S.E., P O BOX 2567, GRAND RAPIDS, MI, 49501, US

(21) Appl. No.: 10/239,013

(22) PCT Filed: Mar. 16, 2001
(86) PCT No.: PCT/US01/08690

(51) Int. Cl. A61K 35/78

(52) U.S. Cl. 424/727

(57) ABSTRACT

A method and composition for the treatment and prevention of hair loss and stimulation of new hair growth by cutting the distal end of hair shafts of the hair; fusing and sealing the cuticle, cortex, and medulla layers of the cut distal end of the hair shaft via a heat-sealing instrument; administering to the scalp or skin and the hair for a sufficient amount of time a sufficient amount of a heated oil composition of at least about 50% coconut oil, preferably at least 75%, and more preferably at least 90% coconut oil.

### BACKGROUND OF THE INVENTION

A. Field of the Invention

The present invention relates to the treatment and prevention of human or animal alopecia conditions.

B. Background of the Art

It is estimated that 35 million Americans experience some degree of alopecia, or hair loss, resulting in 900 million dollars a year being spent in efforts to grow it back. Generally, it affects about 30 million men and 20 million women in the U.S. each year. It is believed that baldness is hereditary and occurs when the hair follicles slowly begin to produce finer and shorter hairs, or stop producing hairs at all. There are many forms of hair loss, ranging from alopecia areata to androgenic alopecia, also known as male or female pattern baldness.

Around 40% of men will have some hair loss by their mid 30's, and nearly 60% of women experience some thinning of the hair after menopause. By age 50, half of all males generally experience hair loss patterns as compared to 50-70% of post-menopausal women. Alopecia becomes more common as age increases, but hair loss may start at younger ages. Without drug therapy or cosmetic treatments, alopecia can remain as a permanent condition.

In addition, animals can also suffer from a variety of dermatologic conditions. Such conditions frequently cause stress to the animal's haircoat resulting in hair loss, which further exacerbates an increased health risk to the animal and increased concern to the pet owner. Common examples of dermatologic conditions of animals, which can result in hair loss, include sebaceous adenitis, primary seborrhea, primary idiopathic seborrhea, seasonal alopecia, and other miscellaneous dermatoses.

To treat human alopecia, the prior art has produced an enormous variety of drug and cosmetic treatments. The reason for such a volume of treatment options stems from the difficulty the medical and scientific community have had in establishing the cause of alopecia. Alopecia has many possible causes, such as genetic disorders, infections, contact with toxic agents, and hormone imbalance. Currently, there are only two drugs approved by the Food and Drug Administration to treat alopecia.

Rogaine®, also known as minoxidil, was originally developed as a treatment for individuals with high blood pressure. While treating those individuals, it was discovered that minoxidil also moderately increased hair growth and prevented future hair loss. It is believed that minoxidil may increase hair growth in the thinning hair area. However, there are problems associated with the use of minoxidil. The most common problems are irritation of the skin, and if the medication is stopped after prolonged use, hair loss may actually increase rather than decrease. Further, major complications of minoxidil are rare, but possible, and patients who suffer from heart disease or hypertension cannot utilize this product without medical supervision. The other approved product to treat alopecia is Propecia®, or finasteride. Propecia® originally was used to treat prostate enlargement by inhibiting the 5-alpha reductase enzyme. Men undergoing that treatment experienced an increase in hair growth while receiving finasteride. It is believed that the inhibition of the 5-alpha reductase enzyme may increase hair follicle growth or prevent future hair follicle loss. However, pregnant or nursing women may not use finasteride because the product is teratogenic, and can harm the human fetus. Due to such a serious health risk, Propecia® has been limited to use within male patient populations only, leaving a majority of female alopecia sufferers to seek alternative treatment options.

Because of the downfalls of currently approved drug therapies for the treatment and prevention of human alopecia conditions, cosmetic treatments have been developed as well. One cosmetic treatment, as noted within U.S. Pat. No. 4,999,187 to Vernon, discloses a hair and scalp treatment composition that comprises 60% by weight or more of petrolatum in combination with 5% by weight or more sulfur, 0.5% by weight or more of 1,2,3-propanetriol, and the remaining 0.5 to 10% of the composition comprised of an oil, such as coconut oil. The composition is said to treat the hair and scalp to effectively prevent dandruff in the hair while conditioning and causing the hair to grow in areas of the scalp where it had previously ceased to grow. However, the '187 patent does not disclose the use of its composition for a variety of alopecia conditions such as alopecia areata. Rather, the '187 patent merely addresses the conditioning of the hair and scalp.

Another cosmetic treatment involves implanting synthetic or artificial hair into hair follicle root bulbs of the scalp, or implanting expandable balloon structures under the scalp, to stimulate natural hair growth. However, it has been found that artificial hair implant methods are almost always unsuccessful. Such implants often become infected, leading to increased patient health risk and further natural hair follicle loss. In response to the failure of artificial hair transplants, other implant treatments utilizing human hair have been developed. Of human transplants available, four invasive surgical methods are currently preferred within the medical community.

The first of those surgical methods includes hair grafts, in which a graft of a small area of hair bearing scalp is moved from one area of the head to another. Alopecia or bald areas are replaced with hair bearing scalp tissue grafts to allow for development of new, natural hair in the transplanted area. However, when such a graft is moved from one area of the scalp to another area, it loses its blood supply, and must reestablish a new one. If a new blood supply cannot be reestablished upon placement of the graft or if infection occurs, this surgical method will fail, resulting in no new hair growth, only scarred tissue.

An alternative surgical method is the hair flap, in which a piece of scalp along with its blood supply is moved from one location on the head to another. This method generally is used only when large areas of hair bearing scalp need to be moved, and the procedure involves a lengthy recovery time. For those seeking a less invasive and shorter recovery time, excision or scalp reduction has been developed. Excision or scalp reduction involves removing the hairless scalp tissue areas and stretching the remaining non-hairless scalp to cover the defect. Here again, the patient is subjected to a surgical procedure with its inherent risks and complications. The last cosmetic surgical treatment method for hair loss involves tissue expansion, in which small balloons are placed beneath the hair bearing scalp, and the balloons are then expanded slowly over several weeks to stretch the adjacent area of the scalp intended to be used to cover the defect. This method again, has surgical risks and complications including scarring, as well as a lengthier treatment and recovery period due to the length of time needed to stretch the existing scalp tissue and for the incisions to heal.

Although surgical cosmetic treatments for alopecia are available, many problems exist with such treatment options. The main problems specifically associated with all of these methods are the need for multiple operations, scarring when the scalp is moved or stretched, unexpected results, and development of numbness in the scalp following the surgical procedure. Further, the patient is subjected to the risks of surgery in general, as well as swelling, bruising, bleeding, and infection during the recovery process.

More than 40 U.S. and several hundred foreign patents have been issued for hair loss/alopecia treatment methods and compositions. Yet, no single agent or method of treatment has been found to universally work against all forms of hair loss. Thus, the treatment process is often one of trial and error, leaving the patient searching for the treatment that will produce the desired result for them.

There is a need in the prior art for a composition and method of treatment regimen for the treatment and prevention of a variety of human alopecia conditions that are non-invasive, can be used in male and female treatment populations without reproductive risk, and can prevent future hair follicle loss while stimulating new hair follicle growth. In addition, there is a need within the prior art for a composition and method of treatment regimen for the treatment and prevention of a variety of animal alopecia conditions to maintain and stimulate a healthy haircoat upon the animal's skin.

### SUMMARY OF THE INVENTION

It has been discovered that human or animal alopecia conditions can be effectively treated or prevented by cutting the hair, heat sealing the cut ends, and applying a heated oil composition before or after the cutting and sealing, preferably comprising at least about 50% coconut oil. These and other features, advantages and objects of the present invention will be further understood and appreciated by those skilled in the art by reference to the following specification and claims. A more detailed description of the present invention shall be discussed further below.

### BRIEF DESCRIPTION OF THE DRAWINGS

FIG. 1 is an illustrative drawing of the anatomy of a human or animal hair seated within and extending from human scalp or animal skin tissue.

FIG. 2 shows growth of the hair shaft from the hair follicle during the anagen stage of growth through the scalp or skin, and also illustrates the natural tapered structural shape of the distal end of a new hair shaft.

FIG. 3 illustrates the natural and structural shape of the distal and proximal ends of a hair after several weeks of growth from the scalp's or skin's surface.

FIG. 4 illustrates the change in the natural and structural shape at the distal end of a hair shaft following cutting of the hair, or wear and tear of the hair through normal daily activities.

FIG. 5 illustrates the re-contouring of the hair shaft to its natural and structural tapered shape at the distal end of the hair, following the cutting step according to the present invention.

FIG. 6 illustrates the sealing and closing of the cuticle, cortex, and medulla layers of the distal end of the hair shaft following the present invention's heat fusion step.

FIG. 7 is a drawing of the hand piece of the apparatus illustrating much of the internal structure of the hand piece.

FIG. 8 is an exploded view of the hand piece.

FIG. 9 is a perspective view of the hand piece.

FIG. 10 is a block diagram of the apparatus.

FIG. 11 is a block diagram of the temperature controller.

### DETAILED DESCRIPTION OF THE PREFERRED EMBODIMENTS

The Method

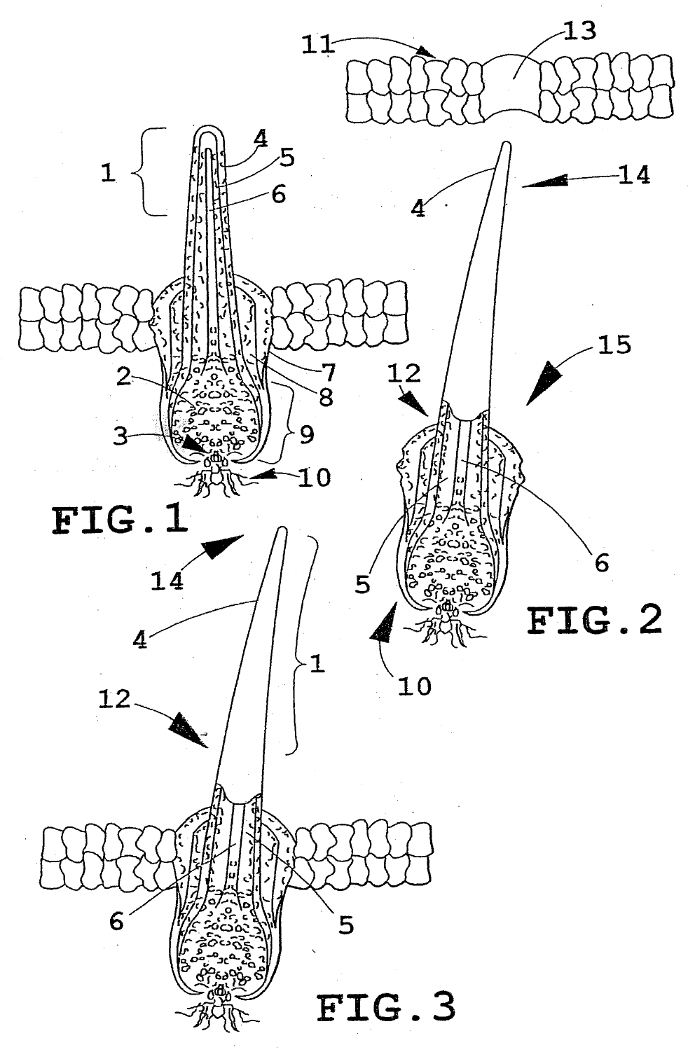

FIG. 1 illustrates the anatomy of a human or animal hair as it is rooted under the scalp or skin by hair bulb 9 and extending through the scalp or skin to expose hair shaft 1. A typical hair comprises of hair shaft 1, which is rooted under the scalp, via dermal papilla 10 attached to hair bulb 9 which is a part of hair follicle 15, as seen in FIG. 2. Further, hair bulb 9 contains melanocyte cells 3 and keratinocyte cells 2 which help form and develop the various layers of hair shaft 1. As hair shaft 1 extends through an opening within the scalp or skin, it extends through external root sheath 7 and internal root sheath 8 which further helps to support and root hair shaft 1 within hair follicle 15 under the scalp or skin. As hair shaft 1 continues to grow, various layers develop, creating defined strata within the overall hair shaft 1. Those layers include an outer layer known as the cuticle layer 4 which surrounds cortex layer 5. Both cuticle layer 4 and cortex layer 5 further surround an internal medulla layer 6. Each of these layers develops from activity of the keratinocyte cells 2 and melanocyte cells 3 within hair bulb 9 of hair follicle 15.

As hair shaft 1 starts to grow, as seen in FIG. 2, towards the opening 13 in scalp or skin 11, the natural or structural shape of the distal end 14 of hair shaft 1 is pointed and closed to cause a tapering of the cuticle layer 4, cortex layer 5, and medulla layer 6. During the growth or anagen stage of a hair, the distal end 14 of hair shaft 1 will continue to remain tapered, such that its diameter will be less than proximal end 12. This can be farther seen in FIG. 3. It is believed that this growth phase for hair follicle 15 to produce hair shaft 1 may last for a period of five years or longer.

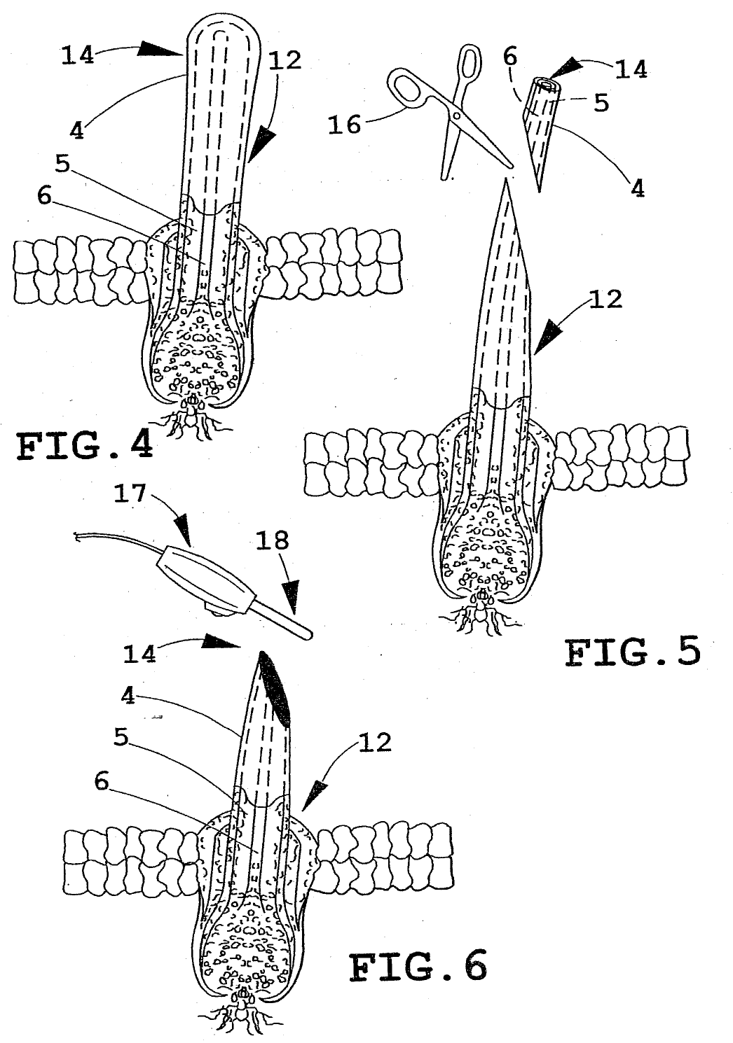

However, during the anagen or growth stage, distal end 14 of hair shaft 1 may lose its closed and pointed tapered shape through hair damage, such as cutting of the hair, or through other daily wear and tear activities that occur over time. FIG. 4 illustrates this loss of tapering for distal end 14 of hair shaft 1, such that the diameter of distal end 14 is equivalent or greater than proximal end 12. As a result, although not being bound to any particular theory, it is believed that cuticle layer 4, cortex layer 5, and medulla layer 6 at distal end 14 lose their closed and pointed shape creating a diameter and weight distribution which will become greater than that of proximal end 12 along hair shaft 1. Soon, the entire hair shaft 1 may lose its natural shape, resulting in a decreased stimulation of keratinocyte cells 2 and melanocyte cells 3 within hair follicle 15 increasing the chance of hair loss due to this anatomical change and growth cell stimulation problem.

While not wishing to be bound by theory, it is believed that sealing and tapering along hair shaft 1, results in increased stimulation of keratinocyte cells 2 and melanocyte cells 3 within hair bulb 9 of hair follicle 15, such that cuticle layer 4, cortex layer 5, and medulla layer 6 continue to grow and taper, decreasing the chance of hair loss from scalp or skin 11. In addition, it is also believed that the method and composition of the present invention reestablishes and lengthens the anagen/growth stage of hair. It is believed that the medulla layer grows at a faster rate than that of the cortex and cuticle layers, which further stimulates and rejuvenates dormant keratinocytes 2 and melanocytes 3 within the hair bulb 9 of the hair follicle 15 to increase production of the cortex and cuticle layers, resulting in an increased proximal end hair shaft diameter, lengthening of the hair shaft, tapering of the distal end of the hair shaft, and extension of the anagen or growth stage for the overall strand of hair. Finally, it is also believed that the method and composition of the present invention could be adapted for animal uses as well.

In one embodiment of the invention, the fusion and heated oil composition application steps are combined such that the fusion step is preferably completed before the heated oil composition application step. In either case, the distal ends of the hair shafts are cut before the fusion step, such that at least the damaged portion of the hair shaft is removed. More preferably, the hair shafts are cut to a length of at least about ¼ to ½ of an inch from the scalp or skin to re-contour the cuticle, cortex, and medulla layers of the distal end of the hair shaft to a tapered shape. This can be seen in FIG. 5, in which cutting instrument 16 is utilized to cut distal end 14 of hair shaft 1, such that cuticle layer 4, cortex layer 5, and medulla layer 6 become tapered.

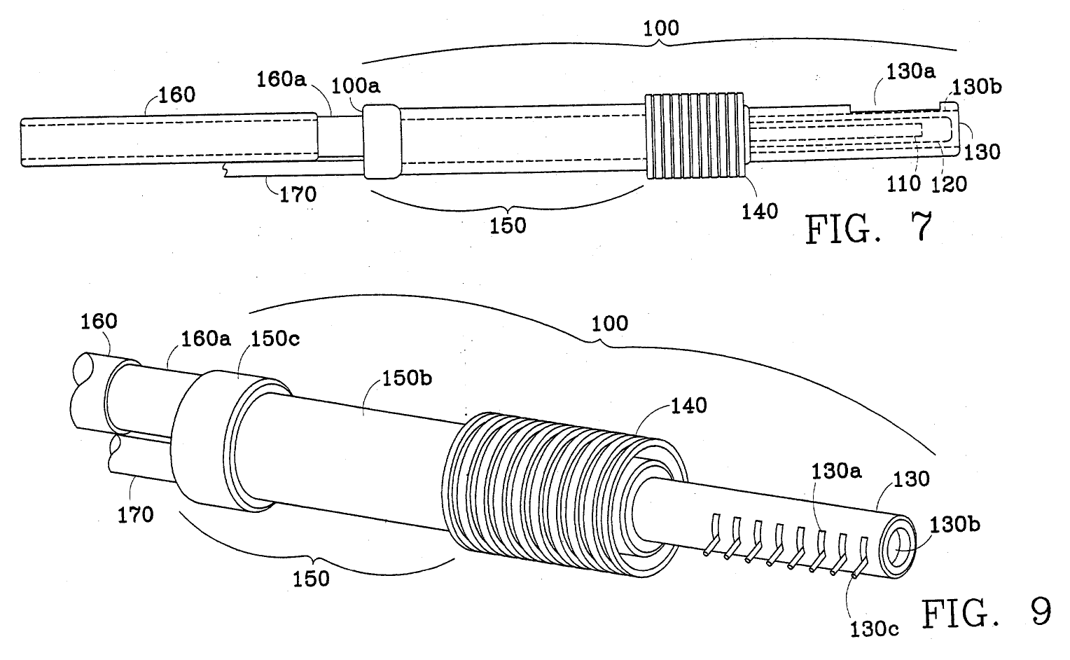

As a result of this cutting, distal end 14 and its cuticle layer 4, cortex layer 5, and medulla layer 6 have a diameter less than proximal end 12 along hair shaft 1. The distal ends of the cut hair shafts are then heat fused and sealed to maintain closure of the cuticle, cortex, and medulla layers to a tapered shape, utilizing a heat-sealing instrument 100 having a heating element 110. Such an instrument seals the cuticle layer 4, cortex layer 5, and medulla layer 6 of hair shaft 1, such that the distal end 14 generally has a diameter less than proximal end 12. This can be seen in FIG. 6.

The temperature utilized to close and seal the distal end of the hair follicle is preferably in the range of about 400° F. to about 1,000° F., depending upon the texture, coarseness, size, and amount of the hair follicles to be treated. The heating instrument which is heated to between about 400° F. to about 1,000° F., preferably between about 600° F. to about 1,000° F., and most preferably between about 800° F. to about 1,000° F., is applied to the distal end of the cut hair shaft for a period of at least about 1-3 seconds to fuse and seal the cuticle, cortex, and medulla layers. As the size, coarseness, and amount of the hair to be treated increases, a higher temperature is required to reestablish the tapered shape of the cuticle, cortex and medulla layers at the distal end of the hair shaft.

A heating device 100 (FIGS. 4, 7-10) is described below, which is currently believed to be the best mode for sealing the ends of the cut hair follicle.

Before or following the cutting and fusing/sealing steps, the hair and scalp or skin are treated with a heated oil composition to further stimulate growth of the existing hair and to stimulate and rejuvenate growth of new hair from dormant hair follicles. The oil composition preferably comprises at least about 50%, more preferably about 75%, and most preferably at least about 90% of a coconut oil component. Most preferably, though not required, at least a portion of the coconut oil is derived from coconuts having one eye, rather than two. Generally, a coconut is comprised of a husk, enclosing a hard shell body which has two or three closely spaced circular markings, commonly referred to as a mouth, from which the sprout will project, and one or two eyes. This hard shell surrounds an inner meat core having a hollow center, which contains a liquid, i.e. coconut juice.

Although not being bound to any particular theory, it is believed that the one-eyed coconut variety produces a coconut oil which is structurally and chemically somewhat different than coconut oil obtained from a two-eyed coconut, resulting in improved, unexpected and surprising hair follicle and hair shaft maintenance and proliferation properties. Thus, coconut oil derived from a two-eyed coconut source can be used within the present invention, but coconut oil derived from a one-eyed coconut source is preferred due to its believed increased hair follicle and hair shaft maintenance and proliferative effects in achieving the desired objectives of the present invention.

To obtain the coconut oil component of the heated oil composition of the present invention, the white liquid-filled meat portion of the coconut is removed from the shell in any conventional manner and then further compressed in any conventional manner to extract the liquid portion, i.e. coconut oil. For example, roughly ten coconuts processed in this manner produce approximately about 100 to 200 milliliters of the extracted liquid from the white meat portion of the coconut, depending upon the size, weight, and age of the coconuts.

Following compression extraction of the liquid from the white coconut meat, the extracted liquid is further combined with the coconut juice of the same coconut and a sufficient quantity of water to allow for further heating and reduction processing. Generally, the amount of coconut juice from ten coconuts is approximately about 50 to 100 milliliters, and the sufficient quantity of water necessary for further reduction processing is approximately about 1 to 2 liters. The combined extracted liquid, coconut juice, and water mixture are then heated to a boiling temperature and reduced to a sufficient temperature at which the mixture will not burn or scorch, and held at that temperature for a period of time of about 2 to 10 hours depending upon the amount of mixture to be reduced to evaporate the water component while producing a refined coconut oil precipitate. Generally, after the coconut liquid extract, coconut juice, and water mixture are brought to a boiling state, the temperature at which the mixture is reduced to form the refined coconut oil precipitate is approximately between about 150° to 300° F. In addition, an aloe vera component may be added to the refined coconut oil precipitate through a heat extraction method discussed below.

After the refined coconut oil precipitate has been produced, the coconut oil is either stored within a conventional refrigerator, not freezer, to prevent microbial damage to the coconut oil, or a preservative is added, in which case refrigeration is not needed.

The oil composition is heated to a temperature of from about 90° F. to about 130° F. prior to administration to the scalp or skin and the hair, and is preferably applied to the scalp or skin and the hair at a temperature of from about 100° F. to about 120° F., and most preferably at a temperature of about 110° F. to about 115° F. The heated oil composition is liberally applied to the scalp or skin and the hair, more preferably in an amount from about 2 to about 20 mL, for a period of time of at least about one to about eight hours before excess of the heated oil composition is removed from the scalp or skin and the hair through conventional shampooing and rinsing.

In an alternative embodiment of the heated oil composition, the heated oil composition may further comprise from about 10% to about 50% aloe vera more preferably also derived from aloe vera plant sources that have been aged for at least six to seven months, and most preferably derived from such aloe vera plant sources, which have not been watered for at least about seven days prior to extraction of the aloe vera component. To properly extract the aloe vera component, the coconut oil component of the oil composition of the present invention is heated to about or above a boiling temperature for a period of about 10 to 45 minutes to heat extract the aloe vera component from the aloe vera plant source when placed within the heated coconut oil component. Such heat extraction forms a composition containing at least about 50% of the coconut oil component and from at least about 10% to about 56% of the aloe vera component. This alternative embodiment of the oil composition is applied in the same manner as the heated oil composition comprised of the coconut oil component alone.

After completion of the administration step, the heated oil composition is then preferably readministered to the scalp or skin, and the hair about three to four additional administrations spaced about one to about two weeks apart, and then reapplied once a month on a continual basis thereafter to continue the desired objectives of the present invention. The heated coconut oil alone or in combination with the heat extracted aloe vera can be reapplied to the scalp or skin and the hair on a more frequent basis as needed to achieve the desired hair shaft and hair follicle maintenance and growth objectives for a particular patient.

In addition to the coconut oil and aloe vera components of the composition, other additional components such as tints, pigments, medicinal agents, conditioners, and the like may be added to the composition without detracting from its effectiveness in treating a variety of alopecia conditions.

The advantage of this fusion/sealing of the distal ends of the hair shafts and application of a heated oil composition and method of treatment is that, in toto, it is painless, non-invasive and been found to be effective in treating a variety of human alopecia conditions, such as alopecia areata, telogen effluvium, and androgenic alopecia. By minimizing the use of invasive procedures and therapeutic side effects of non-naturally occurring agents, a wider male and female patient population can be treated than prior art treatments. In addition, the preferred embodiment can achieve hair growth and hair maintenance results at a substantially greater level and rate than previously achieved with prior art treatments. While again not being limited to any particular theory as to the mode of action of the present invention, these unexpected results are believed to be due to the heated oil composition exerting vasodilatory effects upon the scalp or skin and hair to increase oxygenation and nutrient supplies being delivered to the various growth phase structures, i.e. keratinocytes, melanocytes and other tissue generation cells within the scalp hair follicles, to further stimulate growth and extension to the hair shaft. It is further believed that such vasodilatory effects accomplished through the method and composition of the present invention could be adapted to produce beneficial effects in animals.

It should be further appreciated by those skilled in the art that each of the components of the preferred embodiments can be used separately, but administered generally contemporaneously, and can be given via a cosmetically acceptable dosage form for each component or combination of the components together.

In a further alternative embodiment of the present invention, the heated oil composition comprised of the coconut oil component alone or in combination with the heat extracted aloe vera component can be applied to the scalp or-skin and the hair without the cutting and fusing steps being completed to achieve the desired objectives. For example, the coconut oil component alone can be applied to the scalp or-skin and the hair via a cosmetically acceptable dosage form in a quantity of from about two to about twenty milliliters, which is heated to a temperature of between about 90° F. to about 130° F. prior to being applied to the scalp or skin and hair for about one to about eight hours to stimulate new hair growth while preventing future hair loss.

The Heating Element

In the preferred embodiment, the instrument includes a hand-held device 100 having a heating element 110, a protective shroud 130, and a handle 150. The hand-held device 100 is connected to a control unit 460 that provides electrical power to the heating element 110 and to a vacuum source 420 that draws fumes from the area around the heating element 110. The heating element 110 is heated to a temperature of from about 400° F. to about 1,000° F. sufficient for fusing and sealing the cuticle, cortex, and medulla layers of a hair shaft. The instrument is preferably used after the hair has been cut and either before or after the oil treatment has been applied. The operator sets the heating element to a desired temperature, grips the handle 150 of the hand-held device 100, and maneuvers the heating element 110 to contact the target hair.

In the preferred embodiment, the heating element 110 is enclosed inside a polished chrome plated copper sheath 120 that is thermally coupled to the heating element 110. The sheath 120, not the heating element 110, then contacts the hair to perform the heat-sealing process.

When the hand-held device 100 is in use, various fumes are produced including smoke. An air channel runs through the interior of the hand-held device 100 from the butt of the handle 150 to near the heating element 110. The butt of the hand-held device 100 is connected to the vacuum source thereby drawing fumes from the proximity of the heating element 110, through the handle 150, and through an air hose 160 to the vacuum source 420. As part of the vacuum process, the fumes and air are filtered.

Turning to FIG. 7, the hand-held device 100 of the invention is illustrated in a semi-transparent depiction such that many of the elements are shown assembled together. Hand-held device 100 includes heating element 110, sheath 120, shroud 130, shroud aperture 130A, guard 140, and handle 150. Also illustrated are air hose 160 and electrical cable 170 that connect to the butt 100A of hand-held device 100.

Heating element 110 is a 100 watt resistive type heating element well known to those skilled in the art. The preferred embodiment operates on 24 volts alternating current (VAC) and includes a type K thermocouple type heat sensor built into heating element 110. Four wires exit the base of the heating element 110; two wires for providing electrical power and two wires for the temperature signal from the thermocouple 450. In the prototype, heating element 110 is approximately ⅜ inch in diameter and 5 5/16 inch in length.

Heating element 110 is secured to hand-held device 100 by a hollow cylindrical copper sheath 120. Heating element 110 is positioned inside of sheath 120 which is then threading onto the retainer (not shown) component of hand-held device 100 as illustrated below. Sheath 120 has a polished chrome finish and is thermally coupled to heating element 110 via a thermally conductive paste filling the gap between heating element 110 and the interior of sheath 120. Other surfaces may also be used for sheath 120. For example, it is envisioned that polished metal surfaces, fluoropolymer resins (i.e. TEFLON®), cooking surfaces, and the like will also work well. In the prototype, sheath 12 is approximately ⅝ inches in diameter and 6¼ inches in length.

Sheath 120 and heating element 110 are both positioned inside of shroud 130. Shroud 130 is a substantially hollow cylindrical shaped protective device intended to shield a patient from the high temperatures of heating element 110 and sheath 120. These elements may reach a temperature of about 1,000° F. and can easily burn a patient. Shroud 130 is made of aluminium with a porcelain power coating. A heating element cavity 130B includes the hollow area inside of shroud 130 and houses sheath 120 and heating element 110. Hair-inlet aperture 130A is located on the side of shroud 13 and is sized and shaped to allow hair to enter the heating element cavity 130B while preventing a firm surface (e.g. a patient's scalp) from contacting the high temperature sheath 120 or heating element 110. In alternate embodiments, such as shown in FIG. 9, hair-inlet aperture 130A is comprised of multiple apertures. Shroud 130 threads onto other components of the hand-held device 100 as discussed below.

Adjacent to the base of shroud 130 is guard 140. Guard 140 is another safety/protective feature of the invention that protects the hand of an operator from shroud 130. Even though shroud 130 is generally cooler than heating element 110, it may still be hot enough to bum or to be uncomfortable. Guard 140 is made of high temperature plastic such as garolite and insulates the operator from shroud 130. Guard 140 is secured to handle 150 and has a large guard cavity 140A which allows it to extend over the base of shroud 130 without touching shroud 130.

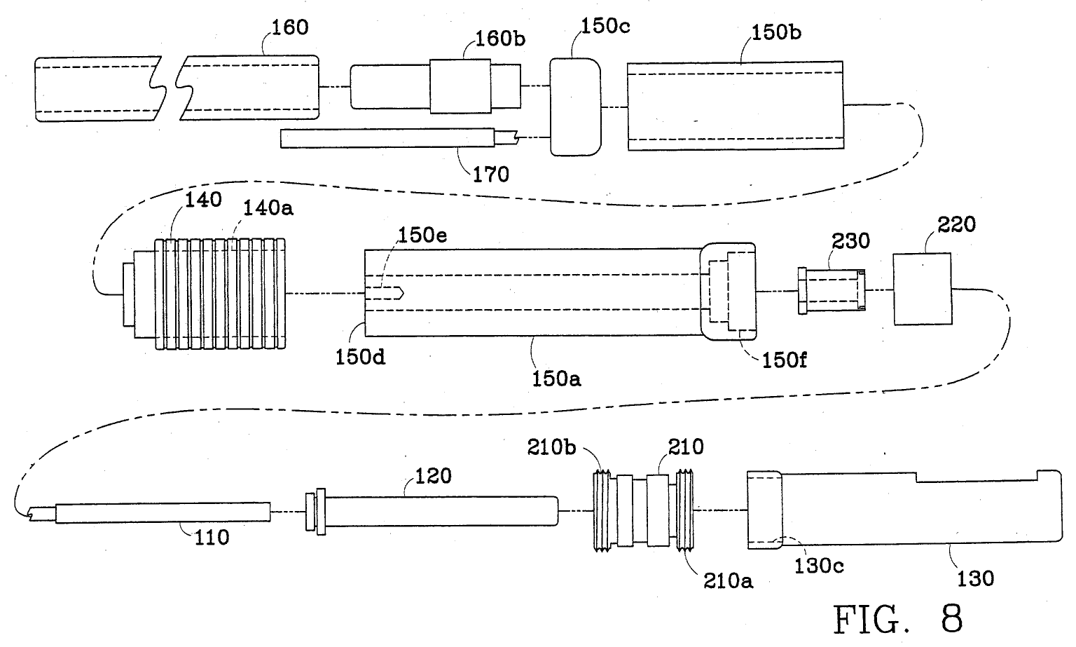

Continuing along FIG. 7, an operator grips the hand-held device 100 via handle 150. Handle 150 includes a plastic frame 150A (see FIG. 8) surrounded by a neoprene rubber cover 150B and an end cap 150C. The plastic frame 150A is substantially hollow and provides a channel for the electrical wires 170 that connect to the heating element 110 and for airflow from the area around heating element 110 to air hose 160. Both the electrical wires 170 and the air hose 160 connect to handle via end cap 150C which threads onto frame 150A.

Air hose 160 is preferably made of a high quality plastic that is able to withstand elevated temperatures. It was found during testing, that a small diameter air hose would become overheated as hot air from the heater element 110 was drawn through the hose. The solution was to increase the diameter of air hose 160 for increased airflow and to use heat tolerant materials. Air hose 160 attaches to hand-held device 100 via coupling 160A.

Electric wires 170 are preferably a high quality electric cable having a braided wire mesh covering the wires. The wire mesh is coated in plastic or similar material and yields a preferred electric cable for the invention.

Referring to FIG. 8 there is shown an exploded view of the hand-held device 100 that shows the components in more detail. Beginning at the bottom of the drawing, shroud 130 threads onto adapter 210 via shroud threads 130C and front adapter threads 210A. This arrangement allows a user to easily remove and clean device 100 as needed. Heating element 110 is inserted into sheath 120, and sheath 120 threads into retainer 230. Ceramic bobbin 220 is clamped between sheath 120 and retainer 230 when sheath 120 and retainer 230 are threaded together. Bobbin 220 has a hole through its center for receiving retainer 230. However, the hole is smaller diameter than the ridge on the base of sheath 120 and bobbin 220 is thereby secured to retainer 230 and sheath 120. Bobbin 220 also provides additional channels for wire 170 from heating element 110 and for airflow drawn from the area proximate to heating element 110. Bobbin 220 serves to insulate the other components from heating element 110.

When sheath 120 is secured to retainer 230, a rigid unit is created comprising the retainer 230, bobbin 220, heating element 110, and sheath 120. This unit is inserted into adapter 210 with sheath 120 extending into the heating element cavity 130B of shroud 130. Adapter 210 is preferably made of aluminium, while retainer 230 is made of steel and bobbin 220 is ceramic.

Continuing with the assembly of the hand-held device 100, the handle frame 150A is treaded onto adapter 210 via rear adapter threads 210B and front frame threads 150F. This secures all of the prior assembled components into a rigid unit. Guard 140 is slid onto the rear 150D of frame 150A and slide forward along handle frame 150A until it stops and guard cavity 140A substantially surrounds the retainer 230, bobbin 220, and the base of shroud 130. Plastic handle frame 150A can still get warm from the hot air drawn through its hollow interior; therefore neoprene rubber cover 150B is slid onto handle frame 150A to provide insulation and a good gripping surface. End cap 150C is then secured to handle frame 150A via two screws threaded into screw holes 150E. The rear of end cap 150C forms the butt 100A of and hand-held device 100. Air hose 160 connects to end cap 150C via connector 160A and electrical wires 170 enter end cap 150C.

Turning to FIG. 9 there is shown a perspective view of hand-held device 100 that also illustrates some alternate embodiments of the invention. An embodiment having multiple hair inlet apertures 130A is shown. Multiple hair inlet apertures may provide better protection against burn injury to a patient; however, it restricts access of hair to the heating element. Another embodiment equips shroud 130 with teeth 130C that function as a comb and are useful in some applications.

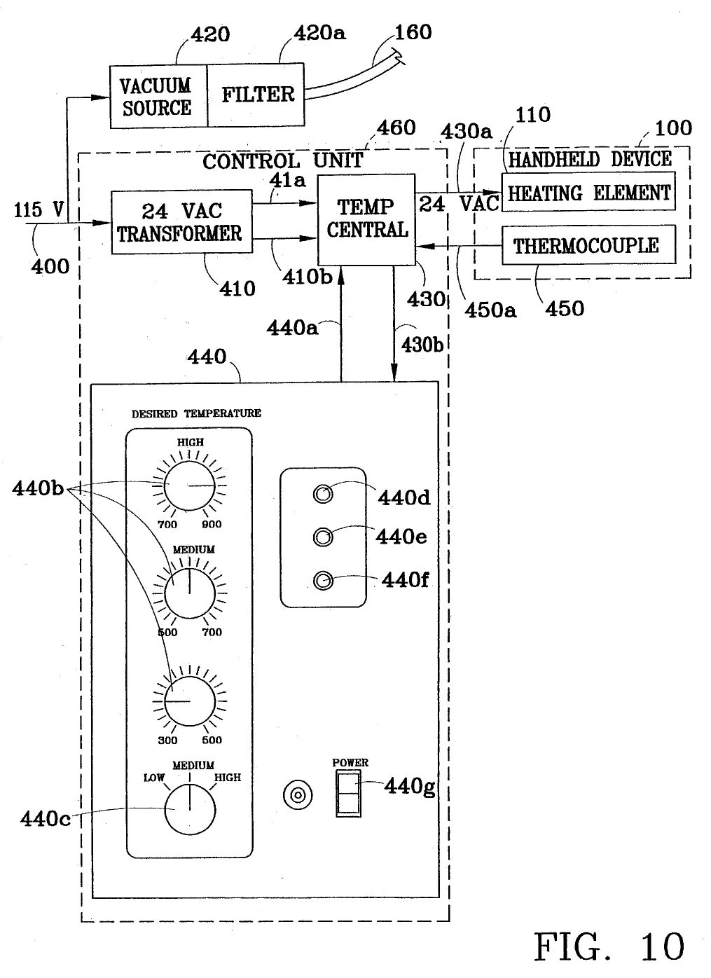

Looking now to FIG. 10, a block diagram of the apparatus is shown. A power source (not shown) provides 115VAC power 400 to medical isolation transformer 410 and vacuum source 420. Medical transformers are often required to power medical devices and are well known in the art. Although it is not required for the operation of the invention, the medical transformer 410 is included to insure compliance with generally accepted standards. Medical transformer 410 provides an isolated 24VAC-power signal 410A to temperature control 430. Power signal 410A is used by temperature control 430 to power heating element 110.

Temperature control 430 controls the temperature of heating element 110. The prototype unit uses a conventional temperature controller such as the PC Programmable Thermocouple Sensing Temperature Controller, model number 5C7-461, manufactured by Oven Industries of Mechanicsburg, Pa. The Operating Manual for the controller is herein incorporated by reference. Those skilled in the art understand that many such temperature control circuits and devices are available and known. The 11.5VAC signal 410B provides power to temperature control 430.

The operation of temperature control 430 is straightforward. An operator enters a desired temperature via user interface 440. User interface 440 generates a temperature command signal 440A representative of the desired temperature and communicates command signal 440A to temperature control 430. The current temperature of heating element 110 is unknown, so temperature signal 450A is communicated from temperature sensor (e.g. thermocouple) 450. Temperature signal 450A provides the feedback data used by temperature control 430 to accurately control the temperature of heating element 110.

In the preferred embodiment, command signal 440A is simply resistance from one of three potentiometers or variable resisters 440B in user interface 440. Each potentiometer 440B has a different resistance range and therefore each potentiometer 440B generates a different temperature command signal. The desired potentiometer is selected using a three-way switch 440C.

Alternate embodiments include, for example, implementing a digital user interface which would generate a digital command signal 440A instead of an analog resistance type signal. Also, thermocouple 450 can be any type of suitable temperature sensor.

Temperature control 430 controls electrical power signal 430A to heating element 110 as a function of command signal 440A from user interface 440 and feedback temperature signal 450A from thermocouple 450. Temperature control 430 communicates a status signal 430B back to user interface 440 which activates one of three lights 440D, 440E and 440F thereby indicating if the measured temperature is above, at, or below the commanded temperature. Lamp 440D is activated if the actual temperature is above the commanded temperature. Similarly, lamp 440F is activated if the actual temperature is below the commanded temperature. If the actual temperature is within a predetermined range specified by the command signal 440A, then lamp 440E is activated. Power switch 440G switches power signal 400 on and off to components of control unit 460.

Temperature control 430 controls the temperature of heating element 110 to between about 400° F. to about 1,000° F., preferably between about 600° F. to about 1,000° F., and most preferably between about 800° F. to about 1,000° F.

Vacuum source 420 is any device capable of drawing a sufficient volume of air through hand-held device 100 to draw fumes from around heating element 110. Vacuum sources are well known in the art. Vacuum source 420 includes a filter 420A for filtering the fumes and particles drawn in by the vacuum source 420. It is preferred to use a high efficiency HEPA filters because of their excellent filtering characteristics. However, other filters may be adequate under certain conditions.

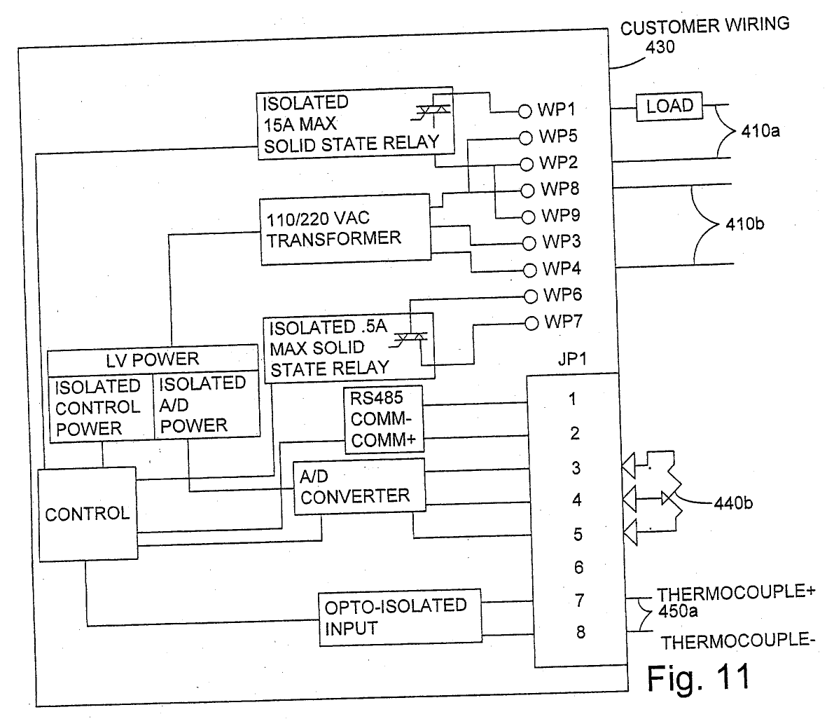

A block diagram of temperature control 430, model number 5C7461 manufactured by Oven Industries, is shown in FIG. 11 connected to other elements of the apparatus. Load or heating element 110 is shown with one lead connected to pin WP1 and a second lead connected to 24 VAC power signal 410A. The circuit is completed when power signal 410A is connected to pin WP2. Control 430 controls the power to heating element 110 via a relay internal to control 430. Power is provided to temperature control 430 via 115 VAC signal 410B which connects to pins WP8 and WP4. Potentiometers or variable resisters 440b connect to pins 3, 4, and 5 of connector JP1. The temperature signal 450A from thermocouple 450 connects to pins 7 and 8 of connector JP1. Temperature controller 430 can be connected to a personal computer or the like in order for a user to set or modify parameters. For example, a user can program the desired control algorithm parameters such as proportional (P), integral (I), and derivative (D), or various combinations of P, I, and D. In the alternative, an ON/OFF mode may be selected with hysteresis.

The hair treatment instrument of the present invention performs the heat sealing process for a method of treating alopecia. The instrument effectively heat-seals the hair of a patient and disposes of fumes generated from the process.

Clinical Results

The alternative embodiments of the present invention were employed on three different groups of patients. The 54 patients' ages ranged from 21 to 74, 22 were females and 32 were males. In all cases, the patients received an initial treatment, followed by at least two subsequent treatments (some received more). In each case, a mixture of 90% coconut oil, 10% aloe vera was used, the aloe vera having been extracted into the coconut oil as described above. The coconut oil used was 70% premium coconut oil and 30% oil from one-eyed coconuts. In some particularly difficult cases, the percentage of one-eyed coconut oil was increased to 50%. The coconut oil was applied at approximately 115° F. In those treatments where sealing was employed, sealing was done at approximately 900° F. The differences in the treatment methods between the three groups is as follows:

Group I: The 17 patients in this group were all treated by first cutting the hair, then sealing, then applying the coconut oil;

Group II: These 17 patients received only oil treatment each time; and

Group III: The 37 patients include the 17 patients from Group I, as well as 20 other patients who received only oil for some of their treatments, and oil combined with sealing for other treatments.

The density and texture of each patients' hair was evaluated at between seven weeks and eighty-one weeks after the first treatment, depending on how long the patient stayed in the study, by three different observers:

The patient himself or herself;

The staff person who conducted the treatment; and

An independent observer who was not advised of the nature of the treatment used on the patient.

Density and texture were measure against a standard established for the Propecia® product, as set forth in product literature. Propecia® has published before and after photographs of results after 24 months showing “no improvement,” “slight improvement,” “moderate improvement,” and “excellent improvement.” These photographs were used by the patients, staff, and independent observer as bench marks for rating the degree of improvement which the patients enjoyed as a result of the Group I, II, or III treatments they received.

The results of the rankings by the patient, staff, and observer are averaged and are shown below in Table 1 (density) and Table 2 (texture). Thus, for example, 5.9% of Group I, 23.5% of Group II, and 16.2% of Group III saw no improvement in hair density. However, 47.1% of Group I, 41.2% of Group II, and 43.2% of Group III saw moderate improvement in hair density.

TABLE 1

| | All subjects | Group I | Group II | Group III |
|---|---|---|---|---|
| DENSITY | (n = 54) | (n = 17) | (n = 17) | (n = 37) |
| No improvement | 18.5 | 5.9 | 23.5 | 16.2 |
| Slight Improvement | 24.1 | 29.4 | 17.6 | 27.0 |
| Moderate Improvement | 42.6 | 47.1 | 41.2 | 43.2 |
| Excellent Improvement | 14.8 | 17.6 | 17.6 | 13.5 |

TABLE 2

| | All subjects | Group I | Group II | Group III |
|---|---|---|---|---|
| TEXTURE | (n = 54) | (n = 17) | (n = 17) | (n = 37) |
| No improvement | 18.5 | 11.8 | 23.5 | 16.2 |
| Slight Improvement | 20.4 | 23.5 | 23.5 | 18.9 |
| Moderate Improvement | 46.3 | 47.1 | 35.3 | 51.4 |
| Excellent Improvement | 14.8 | 17.6 | 17.6 | 13.5 |

The average for all subjects is shown in Tables 1 and 2. 18.5% of all subjects saw no improvement in hair density and no improvement in texture. 24.1% saw slight improvement in density and 20.4% saw slight improvement in texture. 42.6% saw moderate improvement in density and 46.3% saw moderate improvement in texture. Finally, 14.8% saw excellent improvement in density and excellent improvement in texture.

In Table 3, the average results for all subjects, including the average for both density and texture, are compared to the same percentages shown in the Propecia® literature for Propecia® and placebo. As can be seen, the results achieved by the method of the present invention are superior to those achieved either by Propecia® or placebo, in the Propecia® study.

TABLE 3

| | | | The |
|---|---|---|---|
| | Propecia ® | Placebo | Invention |
| No Improvement | 62% | 85% | 18.5% |
| Slight Improvement | 34% | 7% | 22.2% |
| Moderate Improvement | 4% | 0 | 44.4% |
| Excellent Improvement | 0 | 0 | 14.8% |

The above description is that of the preferred embodiments only. Modifications of the invention will occur to those skilled in the art and to those who make or use the invention. Therefore, it is understood that the embodiment described above is merely for illustrative purposes and not intended to limit the scope of the invention, which is defined by the following claims as interpreted according to the principles of patent law, including the Doctrine of Equivalents.

What is claimed is:

1. A method of preventing and treating alopecia conditions by cutting the hair shafts of hair to create a cut distal end on the hair shaft, administering a sufficient amount of heat via a heated instrument to the cut distal end of the hair shaft to seal said cut distal end, and administering to the skin and the hair an effective amount of a heated oil composition, either before or after said cutting and sealing steps.

2. The method of preventing and treating alopecia conditions of claim 1, wherein said oil composition comprises at least about 50% of a coconut oil.

3. The method of preventing and treating alopecia conditions of claim 2, wherein at least a portion of said coconut oil is derived from coconuts having only one eye.

4. The method of preventing and treating alopecia conditions of claim 3, wherein said oil composition is heated to a temperature of from about 90° F. to about 130° F.

5. The method of preventing and treating alopecia conditions of claim 4, wherein said oil composition is heated to a temperature of from about 100° F. to about 120° F.

6. The method of preventing and treating alopecia conditions of claim 5, wherein said oil composition is heated to a temperature of from about 110° F. to about 115° F.

7. The method of preventing and treating alopecia conditions of claim 4, wherein said effective amount of said oil composition is from about 2 to about 20 milliliters, and wherein said sufficient amount of time for which said oil composition is applied to the scalp or skin and the hair is from about one to about eight hours.

8. The method of preventing and treating alopecia conditions of claim 7, wherein said sufficient amount of heat administered to the scalp or skin and the hair via said heated instrument is from about 400° F. to about 1,000° F. and is applied for said sufficient amount of time which is from about one to about three seconds to seal said cut distal end of said hair shaft of said hair.

9. The method of preventing and treating alopecia conditions of claim 8, wherein said sufficient amount of heat administered to the scalp or skin and the hair via said heated instrument is from about 600° F. to about 1,000° F.

10. The method of preventing and treating alopecia conditions of 9, wherein said sufficient amount of heat administered to the scalp or skin and the hair via said heated instrument is from about 800° F. to about 1,000° F.

11. The method of preventing and treating alopecia conditions of claim 8, wherein said oil composition is readministered to the scalp or skin and the hair about 3 to 4 additional administrations spaced about 1 to 2 weeks apart following the initial administration of said oil composition, and then reapplied once a month on a continual basis thereafter.

12. The method of preventing and treating alopecia conditions of claim 8, wherein said hair shafts of said hair are cut to a length where at least about a damaged portion of the distal end of said hair shaft is removed.

13. The method of preventing and treating alopecia conditions of claim 8, wherein said hair shafts of said hair are cut to a length of from about ¼ to about ½ of an inch from the scalp or skin.

14. The method of preventing and treating alopecia conditions of claim 7, wherein said oil composition further comprises from about 10% to about 50% of aloe vera.

15. The method of preventing and treating alopecia conditions of claim 2, wherein said oil composition is heated to a temperature of from about 90° F. to about 130° F.

16. The method of preventing and treating alopecia conditions of claim 15, wherein said oil composition is heated to a temperature of from about 100° F. to about 120° F.

17. The method of preventing and treating alopecia conditions of claim 16, wherein said oil composition is heated to a temperature of from about 110° F. to about 115° F.

18. The method of preventing and treating alopecia conditions of claim 15, wherein said effective amount of said oil composition is from about 2 to about 20 milliliters, and wherein said sufficient amount of time for which said oil composition is applied to the scalp or skin and the hair is from about one about eight hours.

19. The method of preventing and treating alopecia conditions of claim 18, wherein said sufficient amount of heat administered to the scalp or skin and the hair via said heated instrument is from about 400° F. to about 1,000° F. and is applied for said sufficient amount of time which is from about one to about three seconds to seal said cut distal end of said hair shaft of said hair.

20. The method of preventing and treating alopecia conditions of claim 19, wherein said sufficient amount of heat administered to the scalp or skin and the hair via said heated instrument is from about 600° F. to about 1,000° F.

21. The method of preventing and treating alopecia conditions of 20, wherein said sufficient amount of heat administered to the scalp or skin and the hair via said heated instrument is from about 800° F. to about 1,000° F.

22. The method of preventing and treating alopecia conditions of claim 18, wherein said oil composition is comprised of at least about 75% said coconut oil.

23. The method of preventing and treating alopecia conditions of claim 22, wherein at least a portion of said coconut oil is derived from coconuts having only one eye.

24. The method of preventing and treating alopecia conditions of claim 18, wherein said oil composition is comprised of at least about 90% said coconut oil.

25. The method of preventing and treating alopecia conditions of claim 24, wherein at least a portion of said coconut oil is derived from coconuts having only one eye.

26. The method of preventing and treating alopecia conditions of claim 1, wherein said sufficient amount of heat administered to the hair shaft via said heated instrument is from about 400° F. to about 1,000° F. and is applied for said sufficient amount of time which is from about 1 to about 3 seconds to seal said cut distal end of said hair shaft.

27. The method of preventing and treating alopecia conditions of claim 26, wherein said sufficient amount of heat administered to the hair shaft via said heated instrument is from about 600° F. to about 1,000° F.

28. The method of preventing and treating alopecia conditions of 27, wherein said sufficient amount of heat administered to the hair shaft via said heated instrument is from about 800° F. to about 1,000° F.

29. The method of preventing and treating alopecia conditions of claim 26, wherein said oil composition is heated to a temperature of from about 90° F. to about 130° F.

30. The method of preventing and treating alopecia conditions of claim 29, wherein said oil composition is heated to a temperature of from about 100° F. to about 120° F.

31. The method of preventing and treating alopecia conditions of claim 30, wherein said oil composition is heated to a temperature of from about 110° F. to about 115° F.

32. The method of preventing and treating alopecia conditions of claim 28, wherein said effective amount of said oil composition is from about 2 to about 20 milliliters, and wherein said sufficient amount of time for which said oil composition is applied to the scalp or skin and the hair is from about one to about eight hours.

33. The method of preventing and treating alopecia conditions of claim 1, wherein said cutting and sealing steps are performed before said step of administering said heated oil.

34. The method of preventing and treating alopecia conditions of claim 33, wherein said oil composition comprises at least about 50% of a coconut oil.

35. The method of preventing and treating alopecia conditions of claim 34, wherein at least a portion of said coconut oil is derived from coconuts having only one eye.

36. The method of preventing and treating alopecia conditions of claim 33, wherein said oil composition is comprised of at least about 75% of said coconut oil.

37. The method of preventing and treating alopecia conditions of claim 36, wherein at least a portion of said coconut oil is derived from coconuts having only one eye.

38. The method of preventing and treating alopecia conditions of claim 33, wherein said oil composition is comprised of at least about 90% of said coconut oil.

39. The method of preventing and treating alopecia conditions of claim 38, wherein at least a portion of said coconut oil is derived from coconuts having only one eye.

40. A method of preventing and treating alopecia conditions by administering to the scalp or skin and the hair follicles for a sufficient amount of time an effective amount of a heated oil composition comprising at least about 50% coconut oil.

41. The method of preventing and treating alopecia conditions of claim 40, wherein at least a portion of said coconut oil is derived from coconuts having only one eye.

42. The method of preventing and treating alopecia conditions of claim 40, wherein said oil composition is heated to a temperature of from about 90° F. to about 130° F.

43. The method of preventing and treating alopecia conditions of claim 42, wherein said oil composition is heated to a temperature of from about 100° F. to about 120° F.

44. The method of preventing and treating alopecia conditions of claim 43, wherein said oil composition is heated to a temperature of from about 110° F. to about 115° F.

45. The method of preventing and treating alopecia conditions of claim 42, wherein said effective amount of said oil composition is from about 2 to about 20 milliliters.

46. The method of preventing and treating alopecia conditions of claim 42, wherein said sufficient amount of time for which said oil composition is applied to the scalp or skin and the hair is from about one to about eight hours.

47. The method of preventing and treating alopecia conditions of claim 42, wherein said oil composition is readministered to the scalp or skin and the hair about three to about four additional administrations spaced about one to two weeks apart following the initial administration of said oil composition, and then reapplied once a month on a continual basis thereafter.

48. The method of preventing and treating alopecia conditions of claim 42, wherein said oil composition further comprises from about 10% to about 50% of aloe vera.

49. The method of preventing and treating alopecia conditions of claim 40, wherein said oil composition is comprised of at least about 75% said coconut oil.

50. The method of preventing and treating alopecia conditions of claim 40, wherein said oil composition is comprised of at least about 90% said coconut oil.

51. The method of preventing and treating alopecia conditions of claim 50, wherein at least a portion of said coconut oil is derived from coconuts having only one eye.

52. The method of preventing and treating alopecia conditions of claim 40, wherein said oil composition is heated to a temperature of from about 90° F. to about 130° F.

53. The method of preventing and treating alopecia conditions of claim 52, wherein said oil composition is heated to a temperature of from about 100° F. to about 120° F.

54. The method of preventing and treating alopecia conditions of claim 53, wherein said oil composition is heated to a temperature of from about 110° F. to about 115° F.

55. The method of preventing and treating alopecia conditions of claim 52, wherein said effective amount of said oil composition is from about 2 to about 20 milliliters, and wherein said sufficient amount of time for which said oil composition is applied to the scalp or skin and the hair is from about one to about eight hours.

56. A method of preventing and treating alopecia conditions by administering to the scalp or skin and the hair for a period of time of from about one to about eight hours an amount of from about 2 to about 20 milliliters of a heated oil composition which is heated to a temperature of from about 90° F. to about 130° F., wherein said oil composition comprises at least about 50% coconut oil, which is derived from coconuts having only one eye.

57. The method of preventing and treating alopecia conditions of claim 56, wherein said oil composition is heated to a temperature of from about 100° F. to about 120° F.

58. The method of preventing and treating alopecia conditions of claim 57, wherein said oil composition is heated to a temperature of from about 110° F. to about 115° F.

59. The method of preventing and treating alopecia conditions of claim 56, wherein said oil composition further comprises from about 10% to about 50% of aloe vera.

60. The method of preventing and treating alopecia conditions of claim 56, wherein said oil composition is readministered to the scalp or skin and the hair about three to four additional administrations spaced about one to two weeks apart following the initial administration of said oil composition, and then reapplied once a month on a continual basis thereafter.

61. The method of preventing and treating alopecia conditions of claim 56, wherein said oil composition is comprised of at least about 75% said coconut oil.

62. The method of preventing and treating alopecia conditions of claim 56, wherein said oil composition is comprised of at least about 90% said coconut oil.

63. A method of preventing and treating alopecia conditions by cutting the distal ends of hair shafts of the hair to a length of from about ¼ to about ½ of an inch from the scalp or skin; administering heat via a heated instrument at a temperature of from about 400° F. to about 1,000° F. to the cut distal end of said hair shaft for a period of time from about one to about three seconds; and administering to the scalp or skin and the hair for a period of time from about one to about two hours an amount of from about 2 to about 20 milliliters of a heated oil composition, which is heated to a temperature of from about 90° F. to about 130° F., wherein said oil composition comprises at least about 50% coconut oil, which is substantially free of preservatives and derived from coconuts having only one eye.

64. The method of preventing and treating alopecia conditions of claim 63, wherein said heat administered via said heated instrument is from about 600° F. to about 1,000° F.

65. The method of preventing and treating alopecia conditions of claim 64, wherein said heat administered via said heated instrument is from about 800° F. to about 1,000° F.

66. The method of preventing and treating alopecia conditions of claim 63, wherein said oil composition is heated to a temperature of from about 100° F. to about 120° F.

67. The method of preventing and treating alopecia conditions of claim 63, wherein said oil composition is heated to a temperature of from about 110° F. to about 115° F.

68. The method of preventing and treating alopecia conditions of claim 63, wherein said oil composition further comprises at least about 10% of a heat extracted aloe vera.

69. The method of preventing and treating alopecia conditions of claim 63, wherein said oil composition is readministered to the scalp or skin and the hair about three to four additional administrations spaced about one to two weeks apart following the initial administration of said oil composition, and then reapplied once a month on a continual basis thereafter.

70. An oil composition comprising at least about 50% coconut oil derived from coconuts having only one eye.

71. The oil composition of claim 70, wherein said oil composition further comprises at least about 10% of aloe vera.

72. The oil composition of claim 71, wherein said aloe vera is heat extracted.

73. The oil composition of claim 70, wherein said oil composition comprises at least about 75% of said coconut oil.

74. The oil composition of claim 73, wherein said oil composition further comprises at least about 10% of aloe vera.

75. The oil composition of claim 74, wherein said aloe vera is heat.

76. The oil composition of claim 70, wherein said oil composition comprises at least about 90% coconut oil.

77. The oil composition of claim 76, wherein said oil composition further comprises at least about 10% of aloe vera.

78. The oil composition of claim 77, wherein said aloe vera is heat extracted.

79. An oil composition comprising at least about 50% of a coconut oil and at least about 10% of aloe vera.

80. The oil composition of claim 79, wherein said aloe vera is heat extracted.

81. The oil composition of claim 79, wherein at least a portion of said coconut oil is derived-from coconuts having only one eye.

82. The oil composition of claim 79, wherein said oil composition comprises at least about 75% said coconut oil.

83. The oil composition of claim 79, wherein said oil composition comprises at least about 90% said coconut oil.

84. A method of preventing and treating alopecia conditions by cutting the hair shafts of the hair to a length where at least about a damaged portion of the distal end of said hair shaft is removed; administering heat via a heated instrument at a temperature of from about 400° F. to about 1,000° F. to the cut hair shaft at its distal end for a period of time from about one to about three seconds; and administering to the scalp or skin and the hair for a period of time from about one to about eight hours an amount of from about 2 to about 20 milliliters of a heated oil composition, which is heated to a temperature of from about 90° F. to about 130° F., wherein said oil composition comprises at least about 50% coconut oil, which is substantially free of preservatives and derived from coconuts having only one eye.

85. The method of preventing and treating alopecia conditions of claim 84, wherein said heat administered via said heated instrument is at a temperature of from about 600° F. to about 1,000° F.

86. The method of preventing and treating alopecia conditions of claim 85, wherein said heat administered via said heated instrument is at a temperature of from about 800° F. to about 1,000° F.

87. The method of preventing and treating alopecia conditions of claim 84, wherein said oil composition is heated to a temperature of from about 100° F. to about 120° F.

88. The method of preventing and treating alopecia conditions of claim 87, wherein said oil composition is heated to a temperature of from about 110° F. to about 115° F.

89. The method of preventing and treating alopecia conditions of claim 84, wherein said hair shafts of said hair are cut to a length of from about ¼ to about ½ of an inch from the scalp or skin.

90. The method of preventing and treating alopecia conditions of claim 84, wherein said oil composition further comprises at least about 10% of aloe vera.

91. The method of preventing and treating alopecia conditions of claim 90, wherein said aloe vera is heat extracted.

92. The method of preventing and treating alopecia conditions of claim 84, wherein said oil composition is readministered to the scalp or skin and the hair about three to four additional administrations spaced about one to two weeks apart following the initial administration of said oil composition, and then reapplied once a month on a continual basis thereafter.
